(19) United States
(12) Patent Application Publication
(10) Pub. No.: US 2015/0265472 A1
(43) Pub. Date: Sep. 24, 2015

(54) Contoured menstrual sponge

(71) Applicant: Vanessa Givan, Danville, IL (US)

(72) Inventor: Vanessa Givan, Danville, IL (US)

(21) Appl. No.: 14/663,122

(22) Filed: Mar. 19, 2015

Related U.S. Application Data
(60) Provisional application No. 61/955,320, filed on Mar. 19, 2014.

(51) Int. Cl. A61F 13/472

(52) U.S. Cl. CPC A61F 13/47254

(57) ABSTRACT

A contoured menstrual sponge fits between a woman's legs and buttocks. The longitudinal cross section is curved and the lateral cross section is triangular. The sponge is layered, with a core which is composed of relatively thin, collapsed polymeric material which expands upon absorption of a fluid. These collapsed polymeric foam materials are also sufficiently flexible and soft so as to provide a high degree of comfort to the user. The outer layer enables fluid to pass through it to the core while the outer layer remains relatively dry.

### CROSS-REFERENCE TO RELATED APPLICATIONS

This application claims the benefit of U.S. Provisional Application No. 61/955,320, filed Mar. 19, 2014, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to feminine hygiene supplies and accessories, and in particular to a contoured menstrual sponge. For many women, a menstrual period can cause many types of discomfort and anxiety. When seated or laying down, they worry about a possible leakage of menstrual fluid on their clothes, bedding, and furniture upholstery. A woman should be able to move into these various positions to rest without worrying about leaks. Tampons are useful but not always reliable and for some women, using a tampon is uncomfortable or undesirable. A contoured menstrual sponge, which may be used in conjunction with or instead of a tampon, would resolve this problem.

### SUMMARY OF THE INVENTION

Accordingly, the invention is a contoured menstrual sponge. The sponge is intended for use in conjunction with or instead of a tampon, and is designed to fit between a woman's legs and buttocks. The longitudinal cross section is curved and the lateral cross section is triangular. The sponge is layered, with a core which is composed of relatively thin, collapsed polymeric material which expands upon absorption of a fluid. These collapsed polymeric foam materials are also sufficiently flexible and soft so as to provide a high degree of comfort to the user. The outer layer enables fluid to pass through it to the core while the outer layer remains relatively dry.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWING

The accompanying drawing is included to provide a further understanding of the invention and is incorporated into and constitutes a part of the specification. It illustrates one embodiment of the invention and, together with the description, serves to explain the principles of the invention.

FIG. 1 is a side perspective view of the first exemplary embodiment, displaying the sponge 10, the front end 11, and the rear end 12.

FIG. 2 is a top view of the first exemplary embodiment, displaying the sponge 10, the front end 11, and the rear end 12.

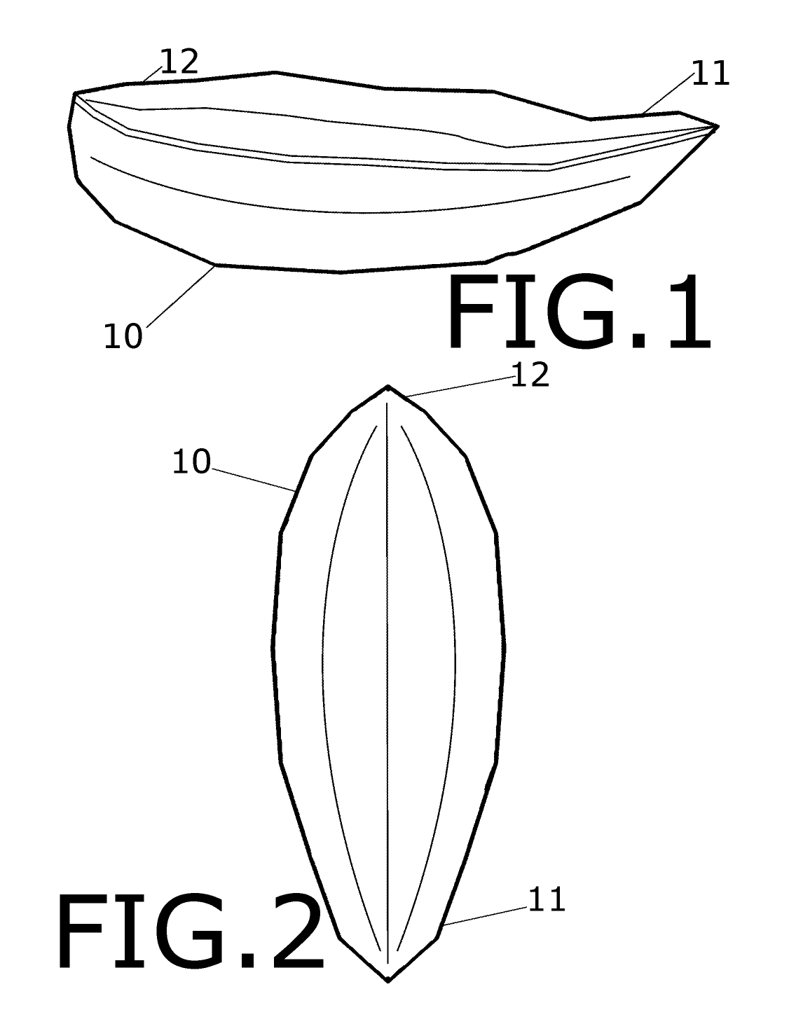

### DETAILED DESCRIPTION OF THE INVENTION

Referring now to the invention in more detail, the invention is directed to a contoured menstrual sponge 10. The sponge 10 is intended for use in conjunction with or instead of a tampon, and is designed to fit between a woman's legs and buttocks. The longitudinal (from the front end 11 to the rear end 12) cross section is curved and the lateral cross section is triangular. The spong is structured with a front end 11 and a rear end 12, the front end being smaller (in terms of cross section) than the rear end. The sponge 10 is layered, with a core which is composed of relatively thin, collapsed polymeric material which expands upon absorption of a fluid. These collapsed polymeric foam materials are also sufficiently flexible and soft so as to provide a high degree of comfort to the user. The outer layer enables fluid to pass through it to the core while the outer layer remains relatively dry.

The first exemplary embodiment comprises a curved sponge 10, with a curved longitudinal cross section and a triangular lateral cross section. The core is composed of absorbent polymeric foam materials which comprise a hydrophilic, flexible, nonionic polymeric foam structure of interconnected open cells. The foam structure has incorporated therein a toxicologically acceptable, hygroscopic, hydrated salt. The chemistry is primarily 2-Ethylhexyl acrylate and divinyl benzene with calcium chloride in the dense layer to maintain the collapsed state.

The collapsed polymeric foam material is obtained by polymerizing a specific type of water-in-oil emulsion, commonly known as High Internal Phase Emulsions or “HIPE.” The oil phase of these HIPE emulsions preferably comprises a monomer component having: (a) a monofunctional glassy monomer; (b) a monofunctional rubbery comonomer; (c) a polyfunctional crosslinking agent component. The oil phase preferably further comprises an emulsifier component which is soluble and will provide a stable emulsion for polymerization. The water or “internal” phase of the HIPE emulsions comprises an aqueous solution containing one or more water-soluble electrolytes. The polymerized foam is subsequently dried to provide the collapsed foam material.

An important aspect of the process is to carry out the emulsion formation and polymerization steps in a manner such that coalescence of the relatively small water droplets formed in the HIPE emulsion is reduced. This leads to an average cell size in the resulting polymeric foam material of approximately 50 microns or less, and is a key mechanism behind manufacturing a consistent quality material. This reduction in coalescence can be consistently achieved either by the use of certain emulsifier systems or either by the use of lower temperatures during polymerization.

The first exemplary embodiment is intended to be disposable after a single use, and is preferably provided in a box which contains a plurality of sponges 10, each individually packaged in paper, plastic or foil. Alternate embodiments, which may provide a single sponge 10 for vending machine sales, are also contemplated.

To use the first exemplary embodiment, a female user follows a method comprising the steps of: unwrapping the sponge 10; inserting the sponge 10 between the user's legs and buttocks; ensuring that the front end 11 faces forward and covers the user's outer labia; and ensuring that the rear end 12 is situated between the user's buttocks. The exterior surface of the sponge 10 is of sufficiently high friction with the user's skin that no attachment or support structure, for example an attachment to the wearer's underwear, is required to retain the sponge 10 in place.

The sponge 10 is preferably manufactured from flexible, durable materials which are lightweight and highly absorbent, such as the collapsed polymeric foam described herein. Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A contoured menstrual sponge comprising a sponge; said sponge having a front end and a rear end; said front end being smaller than said second end; and said sponge being longitudinally curved.

2. The contoured menstrual sponge of claim 1 wherein said sponge is triangular in lateral cross section.

3. The contoured menstrual sponge of claim 1 wherein said sponge lacks any support structure.

4. The contoured menstrual sponge of claim 2 wherein said sponge lacks any support structure.

5. The contoured menstrual sponge of claim 1 wherein said sponge is of high friction with human skin.

6. The contoured menstrual sponge of claim 2 wherein said sponge is of high friction with human skin.

7. The contoured menstrual sponge of claim 3 wherein said sponge is of high friction with human skin.

8. The contoured menstrual sponge of claim 4 wherein said sponge is of high friction with human skin.

9. A method of using a contoured menstrual sponge, the sponge comprising: a sponge; said sponge having a front end and a rear end; said front end being smaller than said second end; and said sponge being longitudinally curved; and the method comprising: a human female user inserting said sponge between said human female user's legs and buttocks; ensuring that said front end faces forward and covers said human female user's outer labia; and ensuring that said rear end is situated between said human female user's buttocks

10. The method of using a contoured menstrual sponge of claim 9 wherein said sponge is triangular in lateral cross section.

11. The method of using a contoured menstrual sponge of claim 9 wherein said sponge lacks any support structure.

12. The method of using a contoured menstrual sponge of claim 10 wherein said sponge lacks any support structure.

13. The method of using a contoured menstrual sponge of claim 9 wherein said sponge is of high friction with human skin.

14. The method of using a contoured menstrual sponge of claim 10 wherein said sponge is of high friction with human skin.

15. The method of using a contoured menstrual sponge of claim 11 wherein said sponge is of high friction with human skin.

16. The method of using a contoured menstrual sponge of claim 12 wherein said sponge is of high friction with human skin.

17. The method of using a contoured menstrual sponge of claim 13 wherein said sponge is of high friction with human skin.
